(19) United States
(12) Patent Application Publication
(10) Pub. No.: US 2025/0027913 A1
(43) Pub. Date: Jan. 23, 2025

(54) SYSTEM AND METHOD FOR ANALYSIS OF EARWAX LIPID PROFILE FOR DISEASE DIAGNOSIS

(71) Applicant: The Research Foundation for the State University of New York, Albany, NY (US)

(72) Inventors: Rabi Musah, Albany, NY (US); Allix Coon, Albany, NY (US); Gavin Setzen, 1400 Washington Avenue, NY (US)

(21) Appl. No.: 18/778,456

(22) Filed: Jul. 19, 2024

Related U.S. Application Data
(60) Provisional application No. 63/527,663, filed on Jul. 19, 2023.

(51) Int. Cl. G01N 30/72; G01N 30/02; G01N 30/88; G01N 33/483; G16H 50/20

(52) U.S. Cl. CPC G01N 30/7206; G01N 33/4833; G16H 50/20; G01N 2030/025; G01N 2030/8813; G01N 2800/14

(57) ABSTRACT

A system and method for analyzing a sample of earwax for chemical components indicating the presence of an otolaryngological disorder, such as Meniere's Disease. The system includes an analyzer that receives an earwax sample from a person, where the analyzer analyzes the earwax sample and produces diagnostic data. The diagnostic data includes chemical component data for the earwax sample and there is a computer device in communication with analyzer, and the computer device receives the diagnostic data from the analyzer and detects a predetermined combination of chemical components in the earwax sample which will indicate the presence of an otolaryngological disorder.

### CROSS-REFERENCE TO RELATED APPLICATION

This invention claims the benefit of U.S. Provisional Patent Application No. 63/527,663, filed Jul. 19, 2023, the entirety of which is hereby incorporated herein by this reference.

### STATEMENT REGARDING FEDERALLY SPONSORED RESEARCH

This invention was made with government support under grant number DC02056501, awarded by the National Institutes of Health. The government has certain rights in the invention.

### BACKGROUND OF THE INVENTION

### 1. Field of the Invention

The present invention generally relates to medical diagnostic systems and methods. More particularly, the present invention relates to a system and method for analysis of the chemical components of earwax samples for purposes of medical diagnoses.

### 2. Description of the Related Art

Mass spectrometry is widely used as a clinical tool for the routine diagnosis of disease and also for the discovery of disease biomarkers. Examples of the use of mass spectrometry for clinical diagnosis include detection of vitamin D deficiencies, bacterial infection, thyroid disease, and some cancers. Currently, one of the most widely accepted clinical uses of mass spectrometry is the newborn bloodspot screening for metabolic disorders including amino acid, acylcarnitine, and fatty acid oxidation dysregulation disorders, organic acidemias, and hemoglobinopathies.

Further, many diseases remain difficult to identify because the occurrence of characteristic biomarkers within traditional biological matrices, such as blood and urine, remain unknown. Disease diagnosis benefits from the analysis of readily accessible, non-traditional matrices that have a high chemical content and contain distinguishing biomarkers. One such matrix is cerumen (i.e., earwax), whose chemical complexity can pose challenges when analyzed by conventional methods. A combination of cerumen chemical profiles can be analyzed by gas chromatography/mass spectrometry (GC-MS) and direct analysis in real time.

Among the classes of illnesses that have not significantly benefited as much from the use of mass spectroscopy are otolaryngologic diseases, which are functional disorders that affect speaking, swallowing, and hearing, among other activities. Meniere's disease is one of the more serious otolaryngologic diseases. It is a chronic, debilitating, and incurable vestibular disorder that is characterized by a recurring set of symptoms that are believed to be the result of abnormally large amounts of endolymph in the inner ear. Its manifestations include unpredictable recurrent episodes of vertigo, tinnitus, imbalance, nausea and/or vomiting, a feeling of fullness or pressure in the ear, and fluctuating, progressive low-frequency hearing loss. Meniere's disease diagnosis involves the painstaking process of excluding other diseases with overlapping symptoms.

For diseases and disorders that have difficulty in diagnosis, the diagnostic workup, is costly and relies on a combination of patient reported anecdotes about the episodic experience of symptoms, the results of MRI screenings, and balance and hearing tests, which can yield results that are far from conclusive. For this reason, alternative approaches to disease diagnosis that utilize molecular biomarkers in traditional biological matrices continue to be explored.

For diagnosing Meniere's Disease several approaches for simplified diagnosis have been tried, such as detecting the depletion of several proteins in blood derived from Meniere's disease patients, the presence of immunoglobulins in the endolymphatic sac luminal fluid (in addition to increased amounts of circulating antibodies), and the presence of mRNAs that are believed to regulate cochlear genes and inflammatory and/or autoimmune pathways. However, there exist a number of challenges to the exploitation of these observations for disease diagnosis.

The problem with using peripheral blood is that the collection is not practical and must occur during surgery. Further, the finding that the blood labyrinthine barrier shows capillary alterations in Meniere's Disease patients in contrast to those of controls, the samples were acquired from the cochlea of the deceased individuals, and diagnosis using this observation is impractical because this fact of the anatomy is not readily accessible antemortem.

Another attempt to diagnose Meniere's Disease utilizes the chemical profile of urine was used as a reporter of the disease. It was found that after ingesting mannitol, patients with Meniere's Disease exhibited a significant increase in urine volume. However, no insight into the chemical changes associated with the disease and this method of diagnosis was revealed.

Thus, it remains highly desirable to identify a readily accessible biological matrix whose chemical makeup can serve as a reporter of Meniere's Disease and other relevant neurotological disorders so that more rapid and accurate diagnosis can be achieved based on assessment of the presence, absence, or change in concentrations of relevant compounds. It is thus to provide a better diagnostic system and method that overcomes the problems of the prior art systems to diagnose disorders and diseases, such as Meniere's Diseases, that the present invention is primarily directed.

### BRIEF SUMMARY OF THE INVENTION

Briefly described, the present system and method analyzes a sample of earwax for chemical components indicating the presence of an otolaryngological disorder, such as Meniere's Disease. The system includes an analyzer that receives an earwax sample from a person and analyzes the earwax sample and produces diagnostic data. The diagnostic data includes chemical component data for the earwax sample, such as a lipid profile, and there is a computer device in communication with analyzer. The computer device receives the diagnostic data from the analyzer and detects a predetermined combination of chemical components in the earwax sample which will indicate the presence of an otolaryngological disorder.

The present invention utilizes earwax (cerumen) samples for disease diagnosis, preferably with GC-MS and ambient mass spectrometric technique direct analysis in real time-high-resolution mass spectrometry (DART-HRMS, either separately or in combination. GC-MS can be used to assess and identify the potential chemical markers that result in differences between those with and without a diagnosis of Meniere's disease. The use of DART-HRMS enables the rapid analysis of a broad range of complex biological matrices.

The present method requires little to no sample preparation steps for qualitative analysis, and samples can be analyzed in their native form. Further, the analysis is rapid and can be completed within only a few seconds, and as such, make it a potentially powerful tool for the routine analysis of earwax.

In one embodiment, the invention provides a system for analyzing a sample of earwax for chemical components indicating the presence of a disease having an analyzer configured to receive an earwax sample from a person, with the analyzer further configured to analyze the earwax sample and produce diagnostic data from the earwax sample. The diagnostic data includes chemical component data for the earwax sample. There is also a computer device in communication with analyzer, with the computer device configured to receive the diagnostic data from the analyzer and detect a predetermined combination of chemical components in the chemical component data indicating the presence of a disease in the person. The predetermined combination of chemical components can indicate the presence of an otolaryngological disorder such as Meniere's Disease.

The analyzer can be a high-resolution mass spectrometer, or gas chromatograph. The analyzer can further be configured to treat the earwax sample with ethyl acetate prior to analysis. In one embodiment, the predetermined combination of chemical components includes fatty acids, and can include at least one or a combination of: cis-9-hexadecenoic acid; cis-10-heptadecenoic acid, and cis-9-octadecenoic acid, palmitoleic acid, and oleic acid.

In an embodiment, the method for analyzing a sample of earwax for chemical components indicating the presence of a disease starts with the steps of receiving an earwax sample from a person at an analyzer, then analyzing the earwax sample, and producing diagnostic data from the earwax sample, with the diagnostic data including chemical component data for the earwax sample. The method continues with receiving, at a computer device, the diagnostic data from the analyzer, and detecting, at the computer device, a predetermined combination of chemical components in the chemical component data thereby indicating the presence of a disease in the person.

The present invention accordingly provides an advantage in the detection of medical diseases and disorders through the minimally invasive analysis of earwax samples when other methods are significantly more inaccurate. Other advantages and features of the present invention will become apparent to one of skill in the art after review of the present application.

### BRIEF DESCRIPTION OF THE DRAWINGS

FIG. 1 is diagram of one embodiment of the system for analyzing a sample of earwax for chemical components.

FIG. 2A is a graph of a gas chromatogram of earwax from a donor without Ménière's disease and pane.

FIG. 2B is a graph of a gas chromatogram of earwax shows from a donor with Ménière's disease.

FIG. 3 is a table of the compounds detected by GC-MS Analysis of ethyl acetate extracts of earwax plugs derived from individual Ménière's Disease and non-Ménière's Disease donors.

FIG. 4A is a graph of the spectral analysis of representative DART mass spectra of earwax from a donor without Ménière's disease.

FIG. 4B is a graph of the spectral analysis of representative DART mass spectra of earwax from a donor with Ménière's disease.

FIG. 5A is a graph of the mass spectra of the earwax analyzed by DART-HRMS in positive-ion mode and screened against the developed statistical model from a donor without Ménière's disease.

FIG. 5B is a graph of the mass spectra of the earwax analyzed by DART-HRMS in positive-ion mode and screened against the developed statistical model from a donor with a confirmed case of Ménière's disease.

FIG. 5C is a graph of the mass spectra of the earwax analyzed by DART-HRMS in positive-ion mode and screened against the developed statistical model from a donor with a tentative diagnosis of Ménière's disease.

FIG. 6 is a table of protonated, monoisotopic masses detected in the DART mass spectra of the non-Ménière's disease samples.

FIG. 7 is a table protonated, monoisotopic masses detected in the DART mass spectra of Ménière's Disease samples.

FIG. 8 is a table of the average concentrations of the indicated fatty acids in Non-Ménière's disease and Ménière's disease samples of earwax, and the calculated confidence level for each.

### DETAILED DESCRIPTION OF THE INVENTION

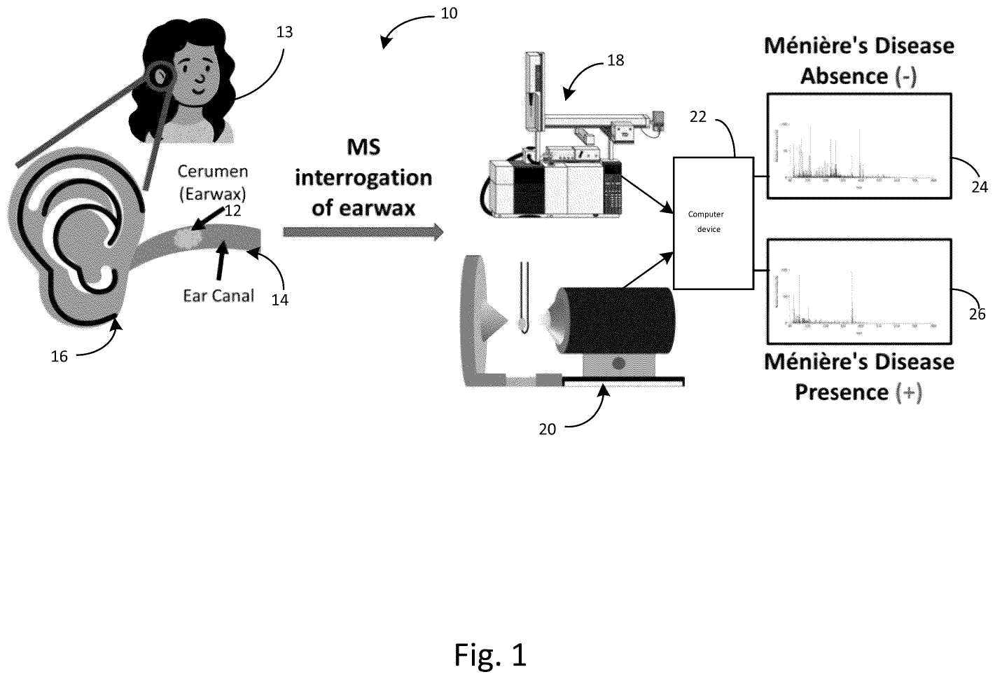

With reference to the figures in which like numerals represent like elements throughout the several views, FIG. 1 is a system 10 for analyzing a sample of earwax 12 from the ear canal 14 of a person 13 for chemical components indicating the presence of a disease. There is an analyzer, such as high-resolution mass spectrometer 20 or gas chromatograph 18 configured to receive an earwax sample 12 from a person 13, with the analyzer further configured to analyze the earwax sample 12 and produce diagnostic data, such as a negative output 24 or positive output 26 from the earwax sample 12. The diagnostic data includes chemical component data for the earwax sample 12, as is further described herein. There is also a computer device 22 in communication with analyzer, e.g. spectrometer 20, with the computer device 22 configured to receive the diagnostic data from the analyzer and detect a predetermined combination of chemical components in the chemical component data indicating the presence of a disease in the person 13, e.g. positive output 26. The predetermined combination of chemical components can indicate the presence of an otolaryngological disorder such as Meniere's Disease. Accordingly, the system 10 can provide a diagnostic method as is more fully described below.

The analyzer can be a high-resolution mass spectrometer 20 or gas chromatograph 18, either singly or in combination. The analyzer can further be configured to treat the earwax sample 12 with ethyl acetate prior to analysis, which is more fully described herein. As is also further described herein, the predetermined combination of chemical components includes fatty acids, and can include at least one or a combination of: cis-9-hexadecenoic acid; cis-10-heptadecenoic acid, and cis-9-octadecenoic acid, palmitoleic acid, and oleic acid.

Cerumen (i.e., earwax), a lipid-rich complex mixture comprising dead skin cells, hair, and various oily secretions produced by the sebaceous and apocrine sweat glands within the ear canal. It contains the most concentrated levels and highest diversity of surface accessible lipids in the human body. There are two types-wet and dry, and the form observed depends on genetics. The type of cerumen produced by an individual is defined by a single nucleotide polymorphism in the ATP-binding cassette C11 gene. Dry-type individuals are homozygous for adenine while the wet-type exhibits at least one guanine. Investigations of the molecular content of cerumen have revealed the presence of a number of compound classes such as or! Panic acids, amino acids, carbohydrates, lipids, alcohols, hydrocarbons, and esters.

The analysis techniques used to detect lipids include gas chromatography (GC), gas chromatography-mass spectrometry (GC-107 MS pyrolysis GC-MS, 4 two-dimensional GC-MS (GC×GC-MS), column chromatography, paper chromatography, and thin layer chromatograph. Even though cerumen is commonly considered to have little to no clinical relevance, its physical characteristics have been found to be associated with several disorders. For example, an increase in waxy constituents is associated with psoriasis; “scanty” and dry earwax is found in those with cystic fibrosis; dark brown or black earwax occurs with alkaptonuria; and those with Parkinson's disease usually produce an excess of wax that can lead to ear canal blockage.

Furthermore, earwax has been shown to contain biomarkers indicative of underlying disease states. The characteristic odor of maple syrup urine disease (MSUD) is detectable in the cerumen of newborns as early as 12 hours after birth. However, in general, the aforementioned physical descriptions cannot be used for definitive diagnosis, and with the exception of MSUD, identification of specific compounds related to these diseases has not been previously accomplished. In 2017, a pilot study was performed comparing earwax from non-diabetic patients and that from donors with type 1 and type 2 diabetes. Statistically significant variations in the concentrations of volatile biomarkers, which enabled discrimination between healthy, type 1, and type 2 diabetic patients, were observed. It was also found that by using headspace GC-MS, patients with and without a diagnosis of certain cancers (carcinoma, lymphoma, and leukemia) could be differentiated from one another using volatile organic markers.

Accordingly, the present invention utilizes the feature that earwax can be used as a disease diagnosis tool and novel approaches could be developed using earwax rather than more traditional body fluids, to screen for diseases. In recent years, technological advances that have been made in the areas of ambient ionization mass spectrometry provide unique opportunities for the investigation of complex lipid-rich matrices such as cerumen and to reveal information about their chemical phenotypes that can be exploited for non-invasive disease diagnosis purposes. In this regard, once a “normal” profile of cerumen is established, there can then be investigations into whether its chemical makeup undergoes changes as a function of different metabolic “states.” These lipid changes are then correlated to various diseases including, but not limited to, inner ear or neurotological disorders and lead to the development of new, rapid, accurate, and non-invasive testing protocols.

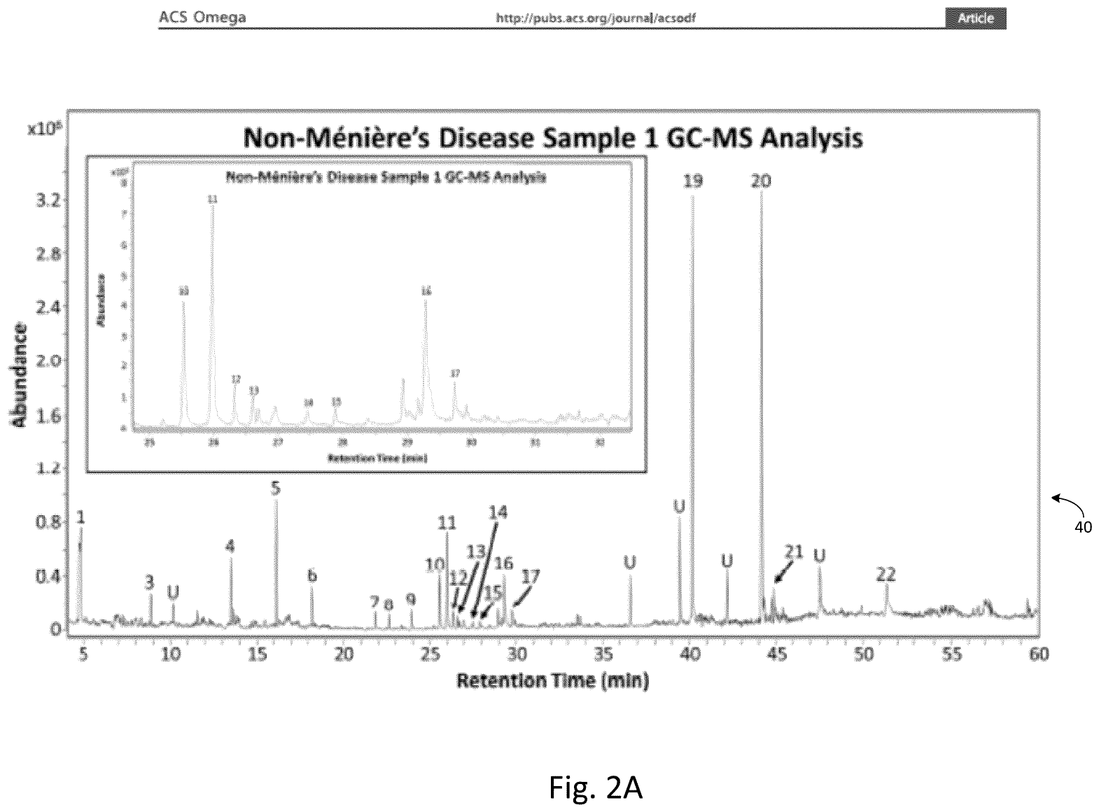

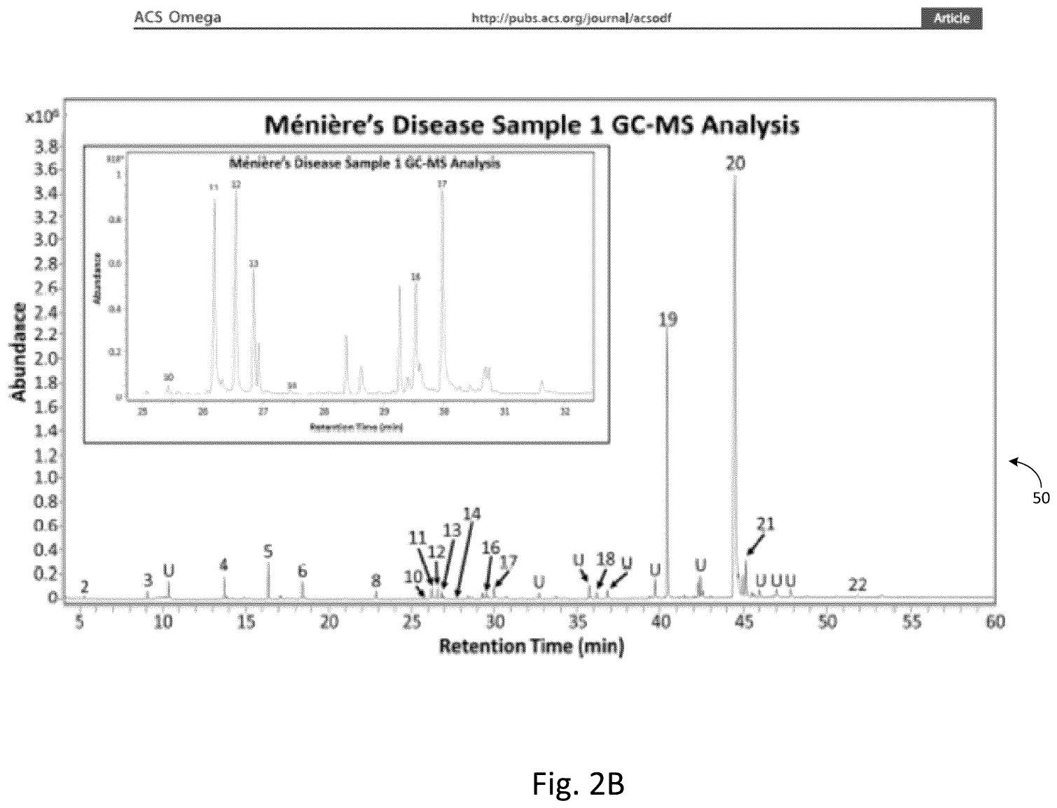

One can also focus on small-molecule biomarkers of Meńiere's disease, revealed by the chemical analysis of cerumen. Because cerumen exhibits high chemical complexity and contains both endogenous and exogenous components that vary depending on donor lifestyle, age, and so forth, the presence of commonalities across Meńie\grave{r}e's disease earwax samples that were lifestyle practices (e.g., personal cleaning of ears or frequent swimming), which contrasted with the chemical signatures of samples from donors who did not have the disease, can be used for diagnostic purposes. To demonstrate this ability, earwax samples (sample 12) were disaggregated into two classes only, purely on the basis of whether or not the donor received a diagnosis of Ménière's disease. To determine whether the chemical profiles of the Ménière's disease and non-Ménière's disease samples exhibited intra-class consistencies and inter-class differences, the chemical profiles of both sample types were first surveyed by gas chromatography-mass spectrometry (GC-MS). It was determined through iterative mass spectrometric analyses of “bulk” cerumen (consisting of earwax plugs from multiple donors) as a function of its suspension in different solvents that ethyl acetate solubilizes the greatest number and broadest range of compounds, and for this reason, ethyl acetate was used for the extractions that were analyzed. Earwax samples for this work were derived from multiple donors as plugs, with each plug representing a single individual. Representative gas chromatograms illustrative of the results of these analyses are presented in FIGS. 2A-2B.

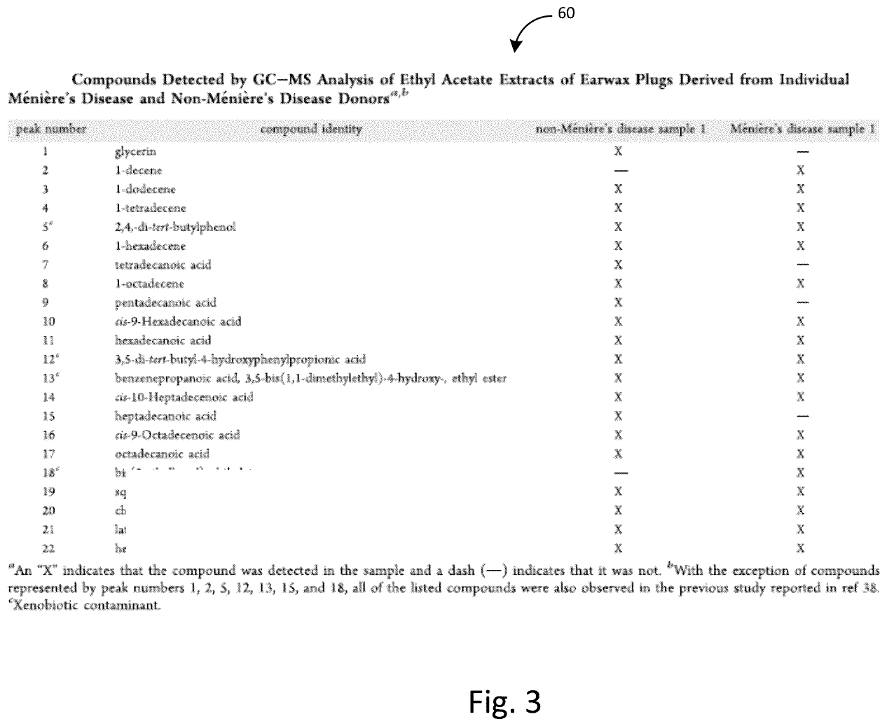

FIG. 2A is a graph 40 of a gas chromatogram of earwax from a donor without Ménière's disease and pane. FIG. 2B is a graph 50 of a gas chromatogram of earwax shows from a donor with Ménière's disease. The identities of the numbered peaks are shown in the table in FIG. 3. Peaks denoted “U” were not identified. The insets highlight the area between 25 and 32 min. FIG. 2A shows the results from a non-Meńie\grave{r}e's disease donor, and panel B shows the results from a donor with a diagnosis of Meńie\grave{r}e's disease. The identities of the numbered peaks, which were assigned based on retention times, mass spectral electron ionization (EI) fragmentation pattern matching, and comparisons to the retention times and fragmentation patterns of authentic standards, are listed in the table in FIG. 3.

FIG. 3 is a table 60 of the compounds detected by GC-MS Analysis of ethyl acetate extracts of earwax plugs derived from individual Ménière's Disease and non-Ménière's Disease donors. In the table, “X” denotes the detection of the indicated compound in the Meńie\grave{r}e's disease or non-Meńie\grave{r}e's disease cerumen sample, and an asterisk (*) indicates exogenous compounds introduced from the environment such as the ubiquitous phthalate plasticizer, bis(2-ethylhexyl)phthalate. The results presented in table 60 also indicate that while most of the identified compounds were detected in both sample classes, a few were not shared. For example, while 1-decene appeared in the Meńie\grave{r}e's disease sample, it was absent in the non-Meńie\grave{r}e's disease sample. However, tetradecanoic acid was detected in the non-Ménière's disease sample, but not the Meńie\grave{r}e's disease sample. It is important to note that there were some instances where tetradecanoic acid was detected in the Ménière's disease earwax plugs, and 1-decene appeared in the non-Ménière's disease samples.

From the chromatograms, a number of trends were apparent. First, all of the molecules observed represented compound classes that have been reported in earlier studies including alkenes, fatty acids, and esters, and all the detected exogenous compounds have been previously reported to be in earwax. Second, the chromatograms of both sample types were dominated by the presence of squalene (peak #19) and cholesterol (peak #20), with the areas under the curves (AUCs) being approximately the same in both samples. For example, the AUC of squalene (peak #19) in the non-Ménière's disease sample (FIG. 2A) was 9.3×106 and in the Ménière's disease sample (FIG. 2B) was 8.3×106. The AUC of cholesterol (peak #20) in the non-Ménière's disease sample (FIG. 2A) was 13.1×106 and in the Ménière's disease sample (FIG. 2B) was 28.7×106. Third, although the chemical profiles of the non-Ménière's disease and Ménière's disease samples were quite similar, it was generally observed that the relative amounts of the molecules present were starkly different, which is readily apparent from visual examination of FIGS. 2A-2B. Relative to squalene and cholesterol, the other peaks in the Ménière's disease chromatogram are diminished in comparison to the chromatogram for the non-Ménière's disease sample. The identities of some of the peaks in the chromatograms are unknown (labeled “U”).

However, even with consideration of these peaks, a fourth observation was that there were no peaks that uniquely appeared in all of the Meńie\grave{r}e's disease samples, and which did not appear in the non-Meńie\grave{r}e's disease samples. This implied that for the compounds detectable by GC-MS, no Meńie\grave{r}e's disease specific biomarkers were observed.

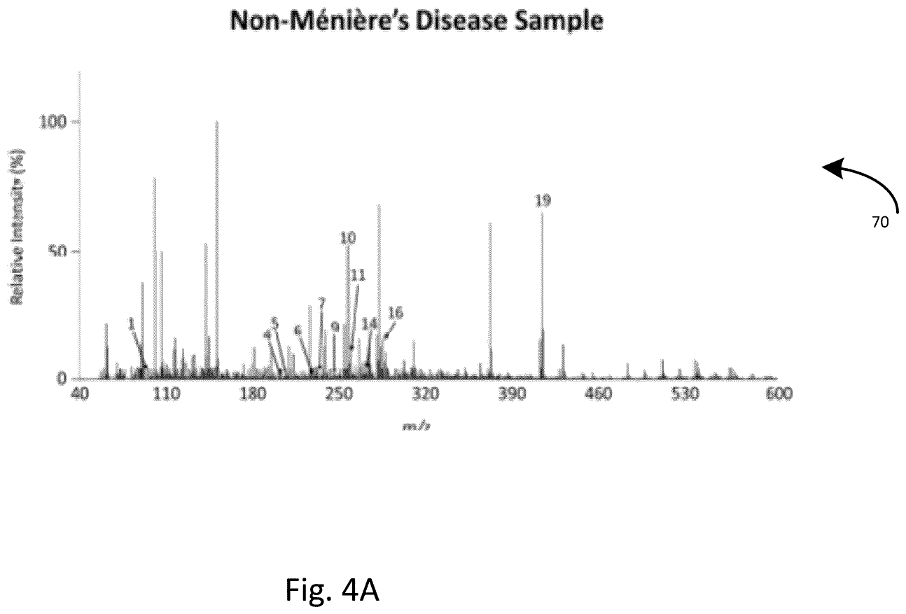

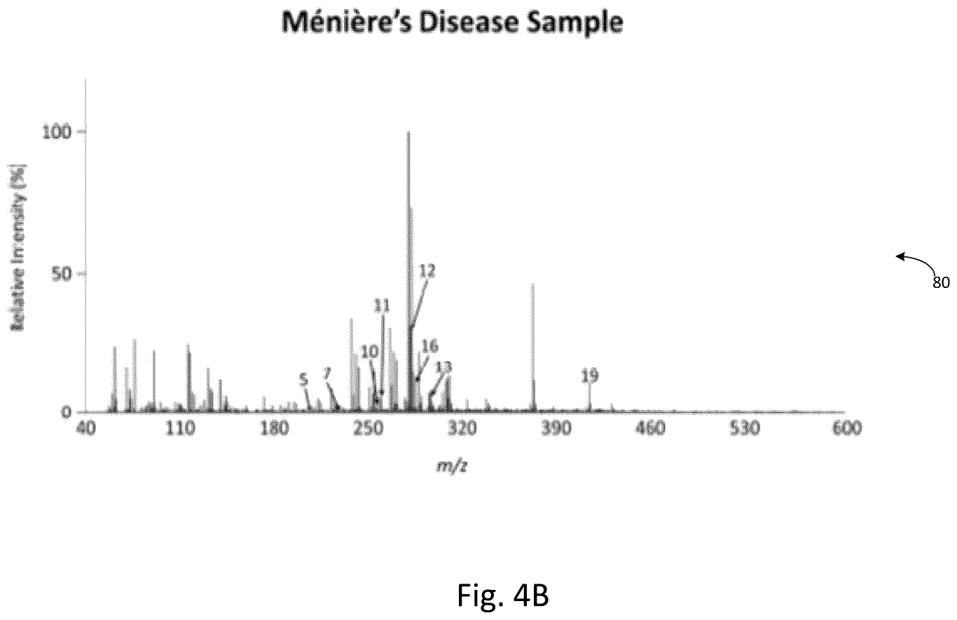

In contrast to the use of GC-MS for interrogation of complex matrices, analyses by DART-HRMS often reveals a broader range of analytes that span the dielectric constant spectrum, that is inclusive of the compounds detected by GC-MS. To determine whether there were other compounds present, over and beyond those detectable by GC-MS and which were unique to Meńie\grave{r}e's disease, earwax was subjected to analysis by DART-HRMS. Earwax samples from fifteen individuals, comprising seven plugs from donors without Meńie\grave{r}e's disease and eight from patients with a confirmed or tentative case of Meńie\grave{r}e's disease were analyzed by DART-HRMS. Representative results for DART mass spectral analysis of cerumen plugs from a non-Meńie\grave{r}e's disease donor and from a patient with a confirmed case of Meńie\grave{r}e's disease are presented in FIGS. 4A-4B, respectively.

FIG. 4A is a graph 70 of the spectral analysis of representative DART mass spectra of earwax from a donor without Ménière's disease. FIG. 4B is a graph 80 of the spectral analysis of representative DART mass spectra of earwax from a donor with Ménière's disease. The identities of the numbered peaks correspond to those identified by GC-MS and are shown in table 60 in FIG. 3. The numbered peaks correspond to protonated monoisotopic high-resolution masses that were consistent with those of compounds confirmed by GC-MS to be present (see table 60, FIG. 3). For example, the peak labeled #19 in the DART mass spectrum in FIG. 4A corresponds to the protonated monoisotopic mass of squalene. Thus, it was observed that there were m/z values in the DART mass spectra that were consistent with the masses of compounds detected by GC-MS. Not every peak identified by GC-MS was detected in every DART mass spectrum, which is why it is important to analyze multiple replicates of each sample. Every peak that was identified by GC-MS had a corresponding m/z value detected by DART-HRMS in at least one replicate of each plug analyzed (data not shown). While we detected masses in the DART mass spectra whose identities were consistent with those found in GC-MS, a broader range of m/z values, over and above those seen in GC-MS, was observed. Detected lipids include hexadecenoic acid, cis-9-hexadecenoic acid, cis-10-heptadece-noic acid, octadecenoic acid, cis-9-octadecenoic acid, squalene, cholesta-3,5-diene, and lanosterol.

The chemical distinctions between these samples may be a consequence of differences in diet, lifestyle, age, or even the presence of medications consumed by the individual. For example, in the analysis of some samples, prescription medications such as sildenafil were observed (data not shown). A range of over 1000 compounds have been detected in earwax. Using a 1% relative abundance threshold cutoff, the number of peaks in the non-Ménière's disease samples ranged from 151 to 775, with the average being 380. This is in stark contrast to the approximately 30 peaks detected by GC-MS and highlights the added benefit of using ambient ionization mass spectrometry as a complementary technique.

In contrast to the range of peaks observed in the non-Meńie\grave{r}e's disease samples, the Meńie\grave{r}e's disease samples exhibited relatively fewer peaks. The average number of peaks in the latter samples was 289, which is ˜24% lower than was observed for the non-Meńie\grave{r}e's disease samples. There is a consistent trend that fewer peaks were observed in the Meńie\grave{r}e's disease samples. It is also similar to what was seen in the GC-MS analyses where the peak areas were reduced. An implication of this is that the Meńie\grave{r}e's disease samples are characterized by a paucity of many of the compounds present in non-Meńie\grave{r}e's disease cerumen, even when accounting for the natural intra-sample variation observed. The observed distinctions between the two were consistently noted in the analyses of all samples.

To determine which subset of the broad range of compounds detected had a statistically significant impact on contrasting between the two classes, the DART mass spectral data were subjected to statistical analysis. Six non-Ménière's disease and six Ménière's disease samples were analyzed. While the utilization of a greater number of samples was desirable, the rarity of Ménière's disease greatly reduced the number available. Thus, even though a far greater number of non-Ménière's disease samples were available, the multivariate statistical analysis processing was limited to the inclusion of only six representative spectra, in order to keep the Ménière's disease and non-Ménière's disease data balanced.

To first determine whether the between class variation was significant in comparison with the within class variations, principal component analysis (PCA) and multivariate analysis of variance (MANOVA) were used. The data were scaled using the “autoscaling” function and subjected to PCA to reduce the dimensionality, and 6 principal components were used in the MANOVA analysis. This process uses multiple variables to estimate the intra- and inter-variation between groups. A p-value of 7.98×10-5 (<0.05, significance threshold level) was obtained by MANOVA, indicating that although there were variations within the mass spectra of the non-Ménière's disease class, and also within the Ménière's disease class samples, the differences between the two classes were statistically significant.

Based on the MANOVA results showing that the DART mass spectra from non-Meńie\grave{r}e's disease and Meńie\grave{r}e's disease donors do exhibit distinct chemical profiles, multivariate statistical analysis was performed. Accordingly, the technique random forest (RF) was applied to the mass spectral data to investigate this trend and identify which m/z values were predictors of Meńie\grave{r}e's disease. A heatmap rendering of the mass spectral data was first generated. From the heatmap rendering, the masses-to-feature selection tool in the Mass Mountaineer software was utilized with a bin width of ±5 milli mass units (mmu) and a relative abundance threshold cutoff of 2% to reveal the masses that were most heavily weighted in facilitating differentiation between the two classes. An iterative process was then employed to reveal the subset of masses that enabled the accurate prediction of the presence of Meńie\grave{r}e's disease. The RF model exhibited a prediction error of 0.2583 381 with modest internal classification merits (accuracy, sensitivity, and precision).

From this treatment, three masses emerged as optimal for the discrimination between the two classes: 255.2324, 269.2481, and 283.2637. These protonated mono-isotopic masses correspond to the formulas C₁₆H₃₀O₂, C₁₇H₃₂O₂, and C₁₈H₃₄O₂ and are consistent with those of three fatty acids confirmed to be present in the cerumen samples by GC-MS analysis and comparison to authentic standards, namely: cis-9-hexadecenoic acid, cis-10-heptadecenoic acid, and cis-9-octadecenoic acid, respectively (appearing in the 25 to 30 min retention time region of the chromatograms).

To assess the ability of the RF model to accurately predict the class of samples that were not included in the creation of the model itself, three external samples were screened against it: one from a donor without Ménière's disease, one from a patient with a confirmed Ménière's disease diagnosis, and one from a patient with a tentative diagnosis of Ménière's disease. Ethyl acetate extracts of these samples prepared as previously described, were each analyzed in replicates of ten.

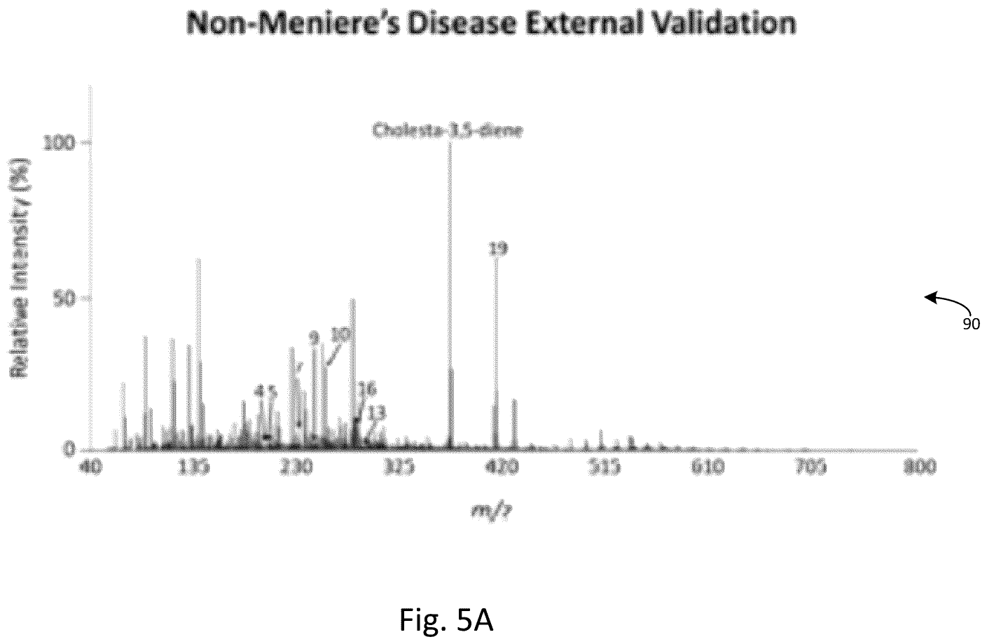

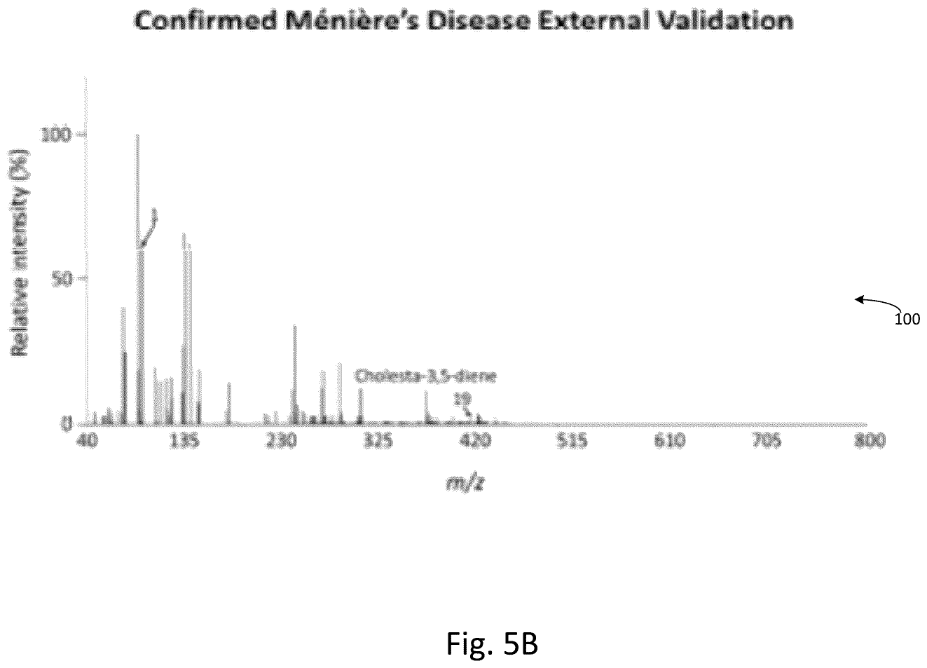

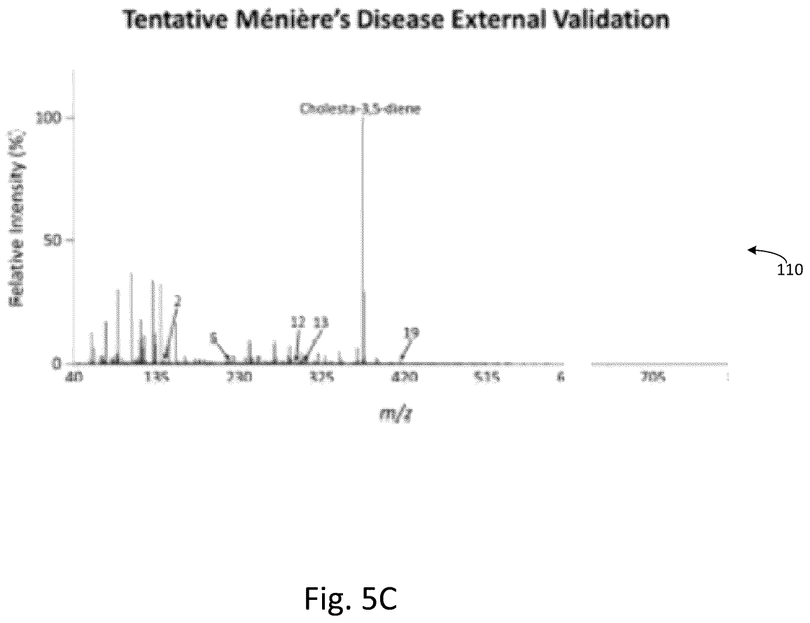

FIG. 5A is a graph 90 of the mass spectra of the earwax analyzed by DART-HRMS in positive-ion mode and screened against the developed statistical model from a donor without Ménière's disease. FIG. 5B is a graph 100 of the mass spectra of the earwax analyzed by DART-HRMS in positive-ion mode and screened against the developed statistical model from a donor with a confirmed case of Ménière's disease. FIG. 5C is a graph 110 of the mass spectra of the earwax analyzed by DART-HRMS in positive-ion mode and screened against the developed statistical model from a donor with a tentative diagnosis of Ménière's disease. The identities of the numbered peaks correspond to those identified by GC-MS and are shown in table 60 of FIG. 3. The compound of cholesta-3,5-diene is also labeled.

FIGS. 5A-5C show a representative spectrum for each of these samples, The identities of the numbered peaks correspond to those identified by GC-MS and are shown in 60 in FIG. 3. The peak corresponding to cholesta-3,5-diene is also labeled. The model was found to be 100% accurate for predicting the class of the external validation samples. The results indicate that: (1) it is highly probable that the patient who received a tentative diagnosis of Meńie\grave{r}e's disease does in fact have the disorder, given that the sample was classified as Meńie\grave{r}e's disease by the RF model. This is supported by the appearance of fewer peaks relative to the large number of peaks visually observed in the spectra of the non-Meńie\grave{r}e's disease samples, and (2) the most likely reason that the external validation results are highly accurate while the classification merits are low is due to the intra-sample disparities between earwax plugs (sample 12) that were members of the same class, as previously described. Although the internal classification results were only modestly accurate, the RF analysis affirmed the utility of the subset of the three fatty acids in enabling discrimination between Meńie\grave{r}e's disease and non-Meńie\grave{r}e's disease samples. In this regard, it was noted that the success in discriminating between the two classes was not a consequence of the presence or absence of the fatty acid biomarkers, but rather a result of the consistently observed lowered ion counts for these compounds in the mass spectra of the earwax of Meńie\grave{r}e's disease donors versus that in the earwax of donors who did not have the disease. Thus, to assess this further, their levels in Ménière's disease and non-Ménière's disease samples were quantified.

To assess whether there were differences in the concentrations of the three fatty acids that appeared to be important in differentiating Ménière's disease and non-Ménière's disease samples, the levels of cis-9-hexadecenoic acid, cis-10-heptadecenoic acid, and cis-9-octadecenoic acid in the two classes of samples were quantified. Nine non-Ménière's disease samples were used, as well as six Meńie\grave{r}e's disease plugs (from 15 individuals in total). The number of Meńie\grave{r}e's disease samples was limited due to disease rarity, but additional non-Meńie\grave{r}e's disease plugs were used to ensure broader representation of samples. In these studies, 19-methyl eicosanoic acid was used as an internal standard. Since this analysis was targeted to detection of fatty acids specifically, hexanes solvent was used in order to maximize their extraction, and a fatty acid specific GC column (HP-FFAP) was used for their detection.

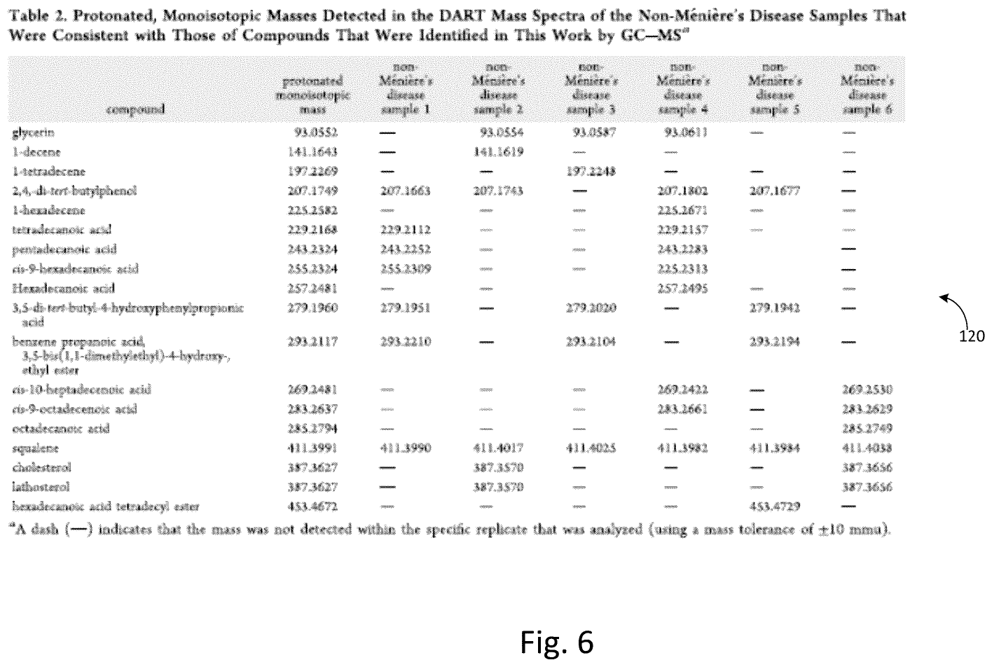

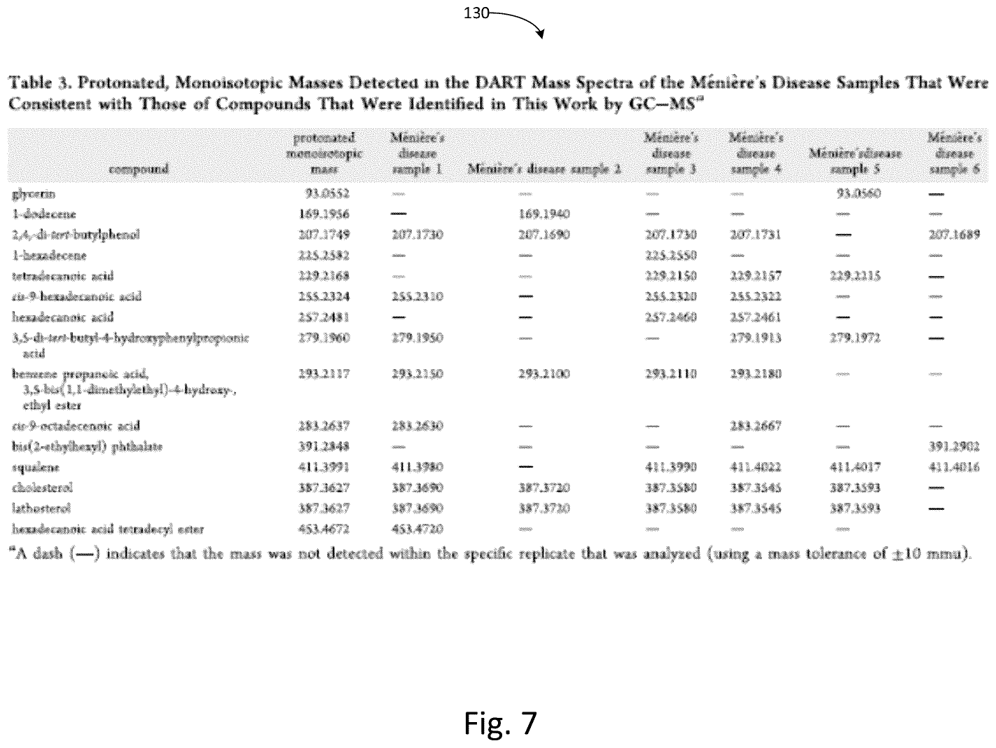

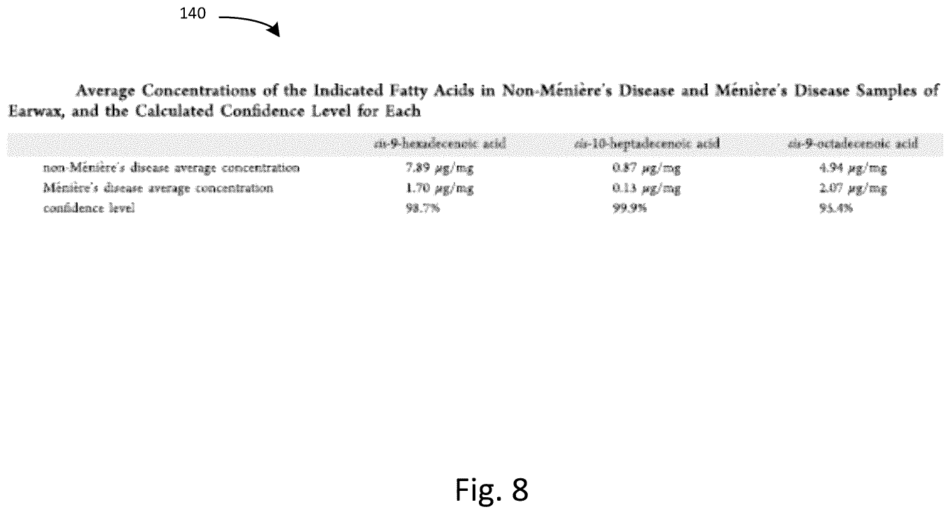

FIG. 6 is a table 120 of protonated, monoisotopic masses detected in the DART mass spectra of the non-Ménière's disease samples. FIG. 7 is a table 130 of protonated, monoisotopic masses detected in the DART mass spectra of Ménière's Disease samples. FIG. 8 is a table 140 of the average concentrations of the indicated fatty acids in Non-Ménière's disease and Ménière's disease samples of earwax, and the calculated confidence level for each.

For the non-Ménière's disease samples, the average concentrations were as follows: cis-9-hexadecenoic acid, 7.89 μg/mg; cis-10-heptadecenoic acid, 0.87 μg/mg; and cis-9-octadecenoic acid, 4.94 μg/mg. For the Ménière's disease samples, the average concentrations were: 462 cis-9-hexadecenoic acid, 1.70 μg/mg; cis-10-heptadecenoic acid, 0.13 μg/mg; and cis-9-octadecenoic acid, 2.07 μg/mg, and these values are reported in table 130 in FIG. 8.

Overall, it was found that compared to the non-Ménière's disease samples, the Ménière's disease samples showed a marked decrease in the concentrations of these three fatty acids of ˜78.4, 85.3, and 58.2%, respectively. The determination of the strength of each of these fatty acid variables in enabling differentiation of the two sample types (i.e., the confidence level) was performed using a T-test. The confidence levels were computed to be: 98.7% for cis-9-hexadecenoic acid; 99.9% for cis-10-heptadecenoic acid; and 95.4% for cis-9-octadecenoic acid (presented in table 130). The observation that the confidence levels for all three fatty acids were above 90% indicates that their ability to enable differentiation of the two sample classes is statistically significant.

To determine whether the levels of the fatty acids in the correctly classified external validation samples in FIGS. 5A-5C aligned with the trends observed in the Ménière's disease and non-Ménière's disease control samples (i.e., lower levels of fatty acids in the former compared to the latter), the quantification of the fatty acids in the external validation samples was also performed. For the external validation non-Ménière's disease sample, the amounts of cis-9-hexadecenoic acid, cis-10-heptadecenoic acid, and cis-9-octadecenoic acid were 19.12, 1.91, and 12.62 μg/mg, respectively. These values are all above the average concentration for these fatty acids in non-Ménière's disease samples. For the external validation tentative Ménière's disease sample, the concentrations of cis-9-hexadecenoic acid, cis-10-heptadecenoic acid, and cis-9-octadecenoic acid were 2.12, 0.48, and 11.49 μg/mg, respectively.

The values of cis-9-hexadecenoic acid and cis-10-heptadecenoic acid were slightly above the average value typical for Ménière's disease but lower than the average values for non-Ménière's disease. The value of cis-9-octadecenoic acid was much higher than even the non-Ménière's disease average. For the external validation confirmed Ménière's disease sample, cis-9-hexadecenoic acid, cis-10-heptadecenoic acid, and cis-9-octadecenoic acid concentrations were 7.56, 0.72, and 2.40 μg/mg, respectively. The concentrations of cis-10-heptadecenoic acid and cis-9-octadecenoic acid were just above the average that was typical for Ménière's disease samples. However, they were lower than the average calculated values of the non-Ménière's disease samples for those specific fatty acids. The concentration of cis-9-hexadecenoic acid aligned more with the average concentration of the non-Ménière's disease sample. The results illustrate that it is important to not only consider the concentration levels of all three of these fatty acids together but to also consider the concentration range for each in assessing Ménière's disease. This variability could be a consequence of concentrations of fatty acids that occur on a continuum that reflects disease progression, the lower concentrations being observed with more advanced stages. The study of the progression of Ménière's disease as a function of the concentration of these fatty acids is the focus of the ongoing work. While we were able to identify a combination of three fatty acids, the relative concentrations of which could be used to predict the presence of Meńie\grave{r}e's disease, it remains unknown why the levels of these three fatty acids serve as disease predictors, or whether these fatty acids might also be predictors of other otolaryngological disorders. The etiology of Meńie\grave{r}e's disease remains unknown, but our findings may provide clues about the connection between disease occurrence and lipid dysregulation. These considerations are the subjects of ongoing investigations.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for analyzing a sample of earwax for chemical components indicating a presence of a disease, comprising:
an analyzer configured to receive an earwax sample from a person, the analyzer further configured to:
analyze the earwax sample; and
produce diagnostic data from the earwax sample, the diagnostic data including chemical component data for the earwax sample; and
a computer device in communication with analyzer, the computer device configured to:
receive the diagnostic data from the analyzer; and
detect a predetermined combination of chemical components in the chemical component data indicating the presence of a disease in the person.

2. The system of claim 1, wherein the analyzer is a high-resolution mass spectrometer.

3. The system of claim 1, wherein the analyzer is a gas chromatograph.

4. The system of claim 1, wherein the analyzer further configured to treat the earwax sample with ethyl acetate prior to analysis.

5. The system of claim 1, wherein the predetermined combination of chemical components includes fatty acids.

6. The system of claim 5, wherein the predetermined combination of chemical components includes at least one or a combination of: cis-9-hexadecenoic acid; cis-10-heptadecenoic acid, and cis-9-octadecenoic acid, palmitoleic acid, and oleic acid.

7. The system of claim 1, wherein the predetermined combination of chemical components indicates the presence of an otolaryngological disorder.

8. The system of claim 1, wherein the predetermined combination of chemical components indicates the presence of Meniere's Disease.

9. A method for analyzing a sample of earwax for chemical components indicating a presence of a disease, comprising:
receiving an earwax sample from a person at an analyzer;
analyzing the earwax sample;
producing diagnostic data from the earwax sample, the diagnostic data including chemical component data for the earwax sample;
receiving, at a computer device, the diagnostic data from the analyzer; and
detecting, at the computer device, a predetermined combination of chemical components in the chemical component data thereby indicating the presence of a disease in the person.

10. The method of claim 9, wherein analyzing the earwax sample is analyzing by high-resolution mass spectrometry.

11. The method of claim 9, wherein analyzing the earwax sample is analyzing by gas chromatography.

12. The method of claim 9, further comprising treating the earwax sample with ethyl acetate prior to analyzing the earwax sample.

13. The method of claim 9, wherein detecting the predetermined combination of chemical components includes detecting fatty acids.

14. The method of claim 13, wherein detecting the predetermined combination of chemical components includes detecting at least one or a combination of: cis-9-hexadecenoic acid; cis-10-heptadecenoic acid, and cis-9-octadecenoic acid, palmitoleic acid, and oleic acid.

15. The method of claim 9, further including indicating the presence of an otolaryngological disorder based upon detecting the predetermined combination of chemical components.

16. The method of claim 9, further including indicating the presence of Meniere's Disease based upon detecting the predetermined combination of chemical components.

17. A system for analyzing a sample of earwax for chemical components indicating a presence of a disease, comprising:
an analyzing means configure to receive an earwax sample from a person, the analyzing means for:
analyzing the earwax sample; and
producing diagnostic data from the earwax sample, the diagnostic data including chemical component data for the earwax sample; and
a computing means in communication with analyzer, the computing means for:
receiving the diagnostic data from the analyzer; and
detecting a predetermined combination of chemical components in the chemical component data indicating the presence of a disease in the person.

18. The system of claim 17, wherein the computing means further for detecting a predetermined combination of fatty acids.

19. The system of claim 17, wherein the computing means further for indicating the presence of an otolaryngological disorder.

20. The system of claim 17, wherein the computing means further for indicating the presence of Meniere's Disease.
